(19) United States
(12) Patent Application Publication
(10) Pub. No.: US 2025/0005772 A1
(43) Pub. Date: Jan. 2, 2025

(54) METHOD AND DEVICE FOR ALIGNING SCAN IMAGES OF 3D SCANNER, AND RECORDING MEDIUM HAVING INSTRUCTIONS RECORDED THEREON

(71) Applicant: MEDIT CORP., Seoul (KR)

(72) Inventor: Jong Cheol RO, Seoul (KR)

(73) Assignee: MEDIT CORP., Seoul (KR)

(21) Appl. No.: 18/700,572

(22) PCT Filed: Oct. 11, 2022
(86) PCT No.: PCT/KR2022/015244

§ 371 Date: Apr. 11, 2024

(30) Foreign Application Priority Data
Oct. 14, 2021 (KR) 10-2021-0136227

(51) Int. Cl. G06T 7/33

(52) U.S. Cl. CPC G06T 7/337; G06T 2207/20081; G06T 2207/20084; G06T 2207/30036

(57) ABSTRACT

The method according to one aspect of the present disclosure is a method being performed by an electronic device comprising at least one processor, the method comprising: acquiring at least one two-dimensional scan image by scanning of the three-dimensional scanner and generating a three-dimensional scan data set regarding a subject's oral cavity based on the acquired at least one two-dimensional scan image, the three-dimensional scan data set comprising multiple three-dimensional coordinate values; generating first plane data corresponding to a virtual occlusal plane; determining multiple reference coordinate values based on the three-dimensional scan data set; generating second plane data corresponding to an occlusal plane of the subject's oral cavity based on the multiple reference coordinate values; and aligning the three-dimensional scan data set on the virtual occlusal plane by matching the first plane data with the second plane data.

### TECHNICAL FIELD

The present disclosure relates to a method for aligning a scan image from a three-dimensional scanner (3D scanner) and, more particularly, to a method for aligning a three-dimensional image of an oral cavity received from a three-dimensional scanner.

### BACKGROUND

In general, in order to acquire information about a patient's oral cavity, a three-dimensional scanner which is inserted into the interior of the patient's oral cavity to acquire images of the interior of the oral cavity may be used. For example, a dentist may insert a three-dimensional scanner into the interior of a patient's oral cavity, scan the patient's teeth, gingiva, and/or soft tissue to acquire multiple two-dimensional images of the patient's oral cavity, and apply a 3D modeling technique to construct a three-dimensional image of the patient's oral cavity by using the two-dimensional images of the patient's oral cavity.

Further, based on the three-dimensional image of the patient's oral cavity, a worker may perform an additional task related to the three-dimensional image, including Dental CAD/CAM work. In some embodiments, in order for the worker to more accurately perform the task, the three-dimensional image of the patient's oral cavity may need to be accurately and consistently positioned on a three-dimensional plane that serves as a reference for the task.

However, in the prior art, regardless of the type of data, a three-dimensional image is simply placed at a predetermined location, or a user performs a dragging motion to place the three-dimensional image at a specific location. Consequently, there were inconveniences such as reduced placement accuracy and time delays.

Accordingly, there has been a growing need in the industry for technologies for more accurately placing a three-dimensional image of a patient's oral cavity in an intended space.

### SUMMARY

The present disclosure provides a technology for aligning a three-dimensional image of an oral cavity received from a three-dimensional scanner (3D scanner) on a virtual occlusal plane.

As one aspect of the present disclosure, a method for aligning a scan image of a three-dimensional scanner may be suggested. The method according to one aspect of the present disclosure is a method being performed by an electronic device comprising at least one processor and at least one memory which stores instructions to be executed by the at least one processor, the method comprising: acquiring at least one two-dimensional scan image by scanning of the three-dimensional scanner and generating a three-dimensional scan data set regarding a subject's oral cavity based on the acquired at least one two-dimensional scan image, the three-dimensional scan data set comprising multiple three-dimensional coordinate values; generating first plane data corresponding to a virtual occlusal plane; determining multiple reference coordinate values based on the three-dimensional scan data set; generating second plane data corresponding to an occlusal plane of the subject's oral cavity based on the multiple reference coordinate values; and aligning the three-dimensional scan data set on the virtual occlusal plane by matching the first plane data with the second plane data.

As one aspect of the present disclosure, an electronic device for aligning a scan image of a three-dimensional scanner may be suggested. The electronic device according to one aspect of the present disclosure is an electronic device comprising a communication circuit communicatively connected to a three-dimensional scanner; a memory; and at least one processor, wherein the at least one processor is configured to: acquire at least one two-dimensional scan image by scanning of the three-dimensional scanner and generating a three-dimensional scan data set regarding a subject's oral cavity based on the acquired at least one two-dimensional scan image, the three-dimensional scan data set comprising multiple three-dimensional coordinate values; generate first plane data corresponding to a virtual occlusal plane; determine multiple reference coordinate values based on the three-dimensional scan data set; generate second plane data corresponding to an occlusal plane of the subject's oral cavity based on the multiple reference coordinate values; and align the three-dimensional scan data set on the virtual occlusal plane by matching the first plane data with the second plane data.

As one aspect of the present disclosure, a non-transitory computer-readable recording medium that records instructions to be executed on a computer for aligning a scan image of a three-dimensional scanner may be suggested. The non-transitory computer-readable recording medium according to one aspect of the present disclosure is a non-transitory computer-readable recording medium recording instructions which, when executed by at least one processor, cause the at least one processor to: acquire at least one two-dimensional scan image by scanning of a three-dimensional scanner and generate a three-dimensional scan data set regarding a subject's oral cavity based on the acquired at least one two-dimensional scan image, the three-dimensional scan data set comprising multiple three-dimensional coordinate values; generate first plane data corresponding to a virtual occlusal plane; determine multiple reference coordinate values based on the three-dimensional scan data set; generate second plane data corresponding to an occlusal plane of the subject's oral cavity based on the multiple reference coordinate values; and align the three-dimensional scan data set on the virtual occlusal plane by matching the first plane data with the second plane data.

According to various embodiments of the present disclosure, a three-dimensional image of an oral cavity acquired by a scanner may be conveniently and quickly aligned on a plane desired by a user. As a result, this has the effect of reducing work time required for aligning the image.

According to various embodiments of the present disclosure, an artificial neural network model may be used to determine an occlusal plane for a three-dimensional image, thereby reducing the time and resources required for aligning the three-dimensional image on a user-desired plane.

### BRIEF DESCRIPTION OF DRAWINGS

FIG. 1 illustrates an acquisition of an image of a patient's oral cavity by using a three-dimensional scanner according to an embodiment of the present disclosure.

FIG. 2A is a block diagram of an electronic device and a three-dimensional scanner according to an embodiment of the present disclosure.

FIG. 2B is a perspective diagram of a three-dimensional scanner according to an embodiment of the present disclosure.

FIG. 3 illustrates a method for generating a three-dimensional image of an oral cavity according to an embodiment of the present disclosure.

FIG. 4 illustrates exemplary plane data corresponding to a virtual occlusal plane according to an embodiment of the present disclosure.

FIG. 5A is an illustrative diagram visually depicting curvature information of each of at least one two-dimensional scan image according to an embodiment of the present disclosure.

FIG. 5B is an illustrative diagram visually depicting size information of each of at least one two-dimensional scan image according to an embodiment of the present disclosure.

FIG. 5C is an illustrative diagram visually depicting shape information of each of at least one two-dimensional scan image according to an embodiment of the present disclosure.

FIG. 6 conceptually illustrates a method for using an artificial neural network model according to an embodiment of the present disclosure.

FIG. 7A illustrates a method by which an electronic device according to an embodiment of the present disclosure generates plane data from a three-dimensional scan data set.

FIG. 7B illustrates a method by which an electronic device according to another embodiment of the present disclosure generates plane data from a three-dimensional scan data set.

FIG. 8 is an illustrative diagram depicting a result of matching plane data corresponding to a virtual occlusal plane and plane data corresponding to an occlusal plane of a subject by an electronic device according to an embodiment of the present disclosure.

FIG. 9 is an illustrative diagram depicting results of aligning a three-dimensional scan data set on a virtual occlusal plane by an electronic device according to an embodiment of the present disclosure.

FIG. 10 is a flowchart of operations of an electronic device according to an embodiment of the present disclosure.

FIG. 11 is a flowchart of operations of an electronic device according to an embodiment of the present disclosure.

FIG. 12 illustrates an application example of a method for aligning a three-dimensional scan data set on a virtual occlusal plane according to an embodiment of the present disclosure.

### DETAILED DESCRIPTION

Embodiments of the present disclosure are illustrated for describing the technical idea of the present disclosure. The scope of the claims according to the present disclosure is not limited to the embodiments described below or to the detailed descriptions of these embodiments.

All technical or scientific terms used in the present disclosure have meanings that are generally understood by a person having ordinary knowledge in the art to which the present disclosure pertains, unless otherwise specified. The terms used in the present disclosure are selected for the purpose of clearer explanation of the present disclosure, and are not intended to limit the scope of claims according to the present disclosure.

The expressions “include,” “provided with,” “have” and the like used in the present disclosure should be understood as open-ended terms connoting the possibility of inclusion of other embodiments, unless otherwise mentioned in a phrase or sentence including the expressions.

A singular expression used in the present disclosure can include meanings of plurality, unless otherwise mentioned, and the same is applied to a singular expression recited in the claims. The terms “first,” “second,” etc. used in the present disclosure are used to distinguish a plurality of elements from one another, and are not intended to limit the order or importance of the relevant elements.

The term “unit” used in the present disclosure means a software element or hardware element, such as a field-programmable gate array (FPGA) or an application specific integrated circuit (ASIC). However, a “unit” is not limited to software and hardware. A “unit” may be configured to be stored in an addressable storage medium or may be configured to run on one or more processors. Therefore, for example, a “unit” may include elements, such as software elements, object-oriented software elements, class elements, and task elements, as well as processors, functions, attributes, procedures, subroutines, segments of program codes, drivers, firmware, micro-codes, circuits, data, databases, data structures, tables, arrays, and variables. Functions provided in elements and “unit” may be combined into a smaller number of elements and “units” or further subdivided into additional elements and “units.”

The expression “based on” used in the present disclosure is used to describe one or more factors that influence a decision, an action of determination, or an operation described in a phrase or sentence including the relevant expression, and this expression does not exclude an additional factor influencing the decision, the action of determination, or the operation.

In the present disclosure, when a certain element is described as being “coupled to” or “connected to” another element, it should be understood that the certain element may be connected or coupled directly to the other element or that the certain element may be connected or coupled to the other element via a new intervening element.

In the present disclosure, artificial intelligence (AI) means a technology that imitates human learning ability, reasoning ability, and perception ability and implements them with a computer, and may include concepts of machine learning and symbolic logic. Machine learning (ML) may include an algorithm technology that classifies or learns features of input data by itself. Artificial intelligence technology is a machine learning algorithm that analyzes input data, learns a result of the analysis, and may make judgments or predictions on the basis of a result of the learning. In addition, technologies that use machine learning algorithms to mimic cognitive and judgmental functions of the human brain may also be understood as a category of artificial intelligence. For example, technical fields of linguistic understanding, visual understanding, inference/prediction, knowledge expression, and motion control may be included.

In the present disclosure, machine learning may refer to a process of training a neural network model by using experience of processing data. It may refer to computer software improving its own data processing capabilities through machine learning. A neural network model is constructed by modeling a correlation between data, and the correlation may be expressed by multiple parameters. The neural network model derives a correlation between data by extracting and analyzing features from given data, and optimizing the parameters of the neural network model by repeating the process may be referred to as machine learning. For example, the neural network model may learn a mapping (correlation) between an input and an output with respect to data given in a form of an input/output pair. Alternatively, even when only input data is given, the neural network model may derive a regularity between given data to learn a relationship therebetween.

In the present disclosure, an artificial intelligence learning model, a machine learning model, or a neural network model may be designed to implement a human brain structure on a computer, and may include multiple network nodes that simulate neurons of a human neural network and have weights. The multiple network nodes may have a connection relationship therebetween by simulating synaptic activities of neurons that transmit and receive a signal through synapses. In the artificial intelligence learning model, the multiple network nodes may transmit and receive data according to a convolution connection relationship while being located in layers of different depths. The artificial intelligence learning model may include, for example, an artificial neural network model, a convolution neural network model, and the like.

Hereinafter, embodiments of the present disclosure will be described with reference to the accompanying drawings. In the accompanying drawings, identical or corresponding elements are indicated by identical reference numerals. In the following description of embodiments, repeated descriptions of the identical or corresponding elements will be omitted. However, even when a description of an element is omitted, such an element is not intended to be excluded in an embodiment.

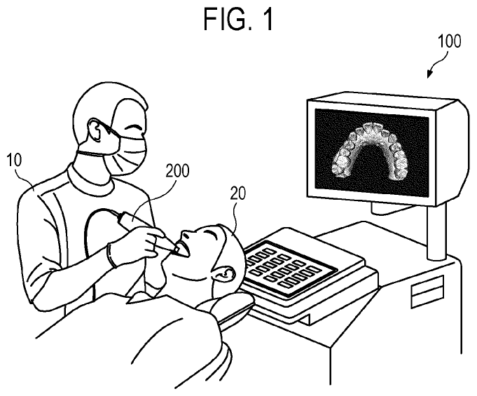

FIG. 1 illustrates an acquisition of an image of a patient's oral cavity by using a three-dimensional scanner 200 according to an embodiment of the present disclosure. According to various embodiments, the three-dimensional scanner 200 may be a dental medical device for acquiring an image of an oral cavity of a subject 20. For example, the three-dimensional scanner 200 may be an intraoral scanner. As illustrated in FIG. 1, a user 10 (e.g., a dentist or a dental hygienist) may use the three-dimensional scanner 200 to acquire an image of the oral cavity of the subject 20 (e.g., a patient) from the subject 20. In another example, the user 10 may acquire an image of the oral cavity of the subject 20 from a diagnostic model (e.g., a plaster model or an impression model) that is made in the shape of the oral cavity of the subject 20. Hereinafter, for ease of description, acquiring an image of the oral cavity of the subject 20 by scanning the oral cavity of the subject 20 will be described. However, the present disclosure is not limited thereto, and it is also possible to acquire an image of another part of the subject 20 (e.g., an ear of the subject 20). The three-dimensional scanner 200 may be shaped to be inserted into or removed from the oral cavity, and may be a handheld scanner which has a scanning distance and a scanning angle freely adjustable by the user 10.

The three-dimensional scanner 200 according to various embodiments may be inserted into the oral cavity of the subject 20 and scan the interior of the oral cavity in a non-contact manner, thereby acquiring an image of the oral cavity. The image of the oral cavity may include at least one tooth, gingiva, or artificial structures insertable into the oral cavity (e.g., orthodontic devices including brackets and wires, implants, dentures, and orthodontic aids inserted into the oral cavity). The three-dimensional scanner 200 may use a light source (or a projector) to emit light to the oral cavity of the subject 20 (e.g., at least one tooth or gingiva of the subject 20), and may receive light reflected from the oral cavity of the subject 20 through a camera (or at least one image sensor). According to another embodiment, the three-dimensional scanner 200 may scan a diagnostic model of the oral cavity to acquire an image of the diagnostic model of the oral cavity. When the diagnostic model of the oral cavity is a diagnostic model that mimics the shape of the oral cavity of subject 20, an image of the diagnostic model of the oral cavity may be an image of the oral cavity of the subject. For ease of description, the following description assumes, but is not limited to, acquiring an image of the oral cavity by scanning the interior of the oral cavity of the subject 20.

The three-dimensional scanner 200 according to various embodiments may acquire a surface image of the oral cavity of the subject 20 as a two-dimensional image based on information received through a camera. The surface image of the oral cavity of subject 20 may include at least one among at least one tooth, gingiva, artificial structure, cheek, tongue, or lip of the subject 20. The surface image of the oral cavity of subject 20 may be a two-dimensional image.

The two-dimensional image of the oral cavity acquired by the three-dimensional scanner 200 according to various embodiments may be transmitted to an electronic device 100 that is connected via a wired or wireless communication network. The electronic device 100 may be a computer device or a portable communication device. The electronic device 100 may generate a three-dimensional image (or, a three-dimensional oral cavity image or a three-dimensional oral model) of the oral cavity, which is a three-dimensional representation of the oral cavity, based on the two-dimensional image of the oral cavity received from the three-dimensional scanner 200. The electronic device 100 may generate the three-dimensional image of the oral cavity by modeling the inner structure of the oral cavity in three dimensions, based on the received two-dimensional image of the oral cavity.

The three-dimensional scanner 200 according to another embodiment may scan the oral cavity of the subject 20 to acquire a two-dimensional image of the oral cavity, generate a three-dimensional image of the oral cavity based on the acquired two-dimensional image of the oral cavity, and transmit the generated three-dimensional image of the oral cavity to the electronic device 100.

The electronic device 100 according to various embodiments may be communicatively connected to a cloud server (not shown). In this case, the electronic device 100 may transmit a two-dimensional image of the oral cavity of the subject 20 or a three-dimensional image of the oral cavity to the cloud server, and the cloud server may store the two-dimensional image of the oral cavity of the subject 20 or the three-dimensional image of the oral cavity, which has been received from the electronic device 100.

According to another embodiment, in addition to a handheld scanner which is inserted into the oral cavity of subject 20 and used, a table scanner (not shown) which is fixed and used in a specific location may be used as the three-dimensional scanner. The table scanner may generate a three-dimensional image of a diagnostic model of the oral cavity by scanning the diagnostic model of the oral cavity. In the above case, a light source (or a projector) and a camera of the table scanner are fixed, allowing the user to scan the diagnostic model of the oral cavity while moving the diagnostic model of the oral cavity.

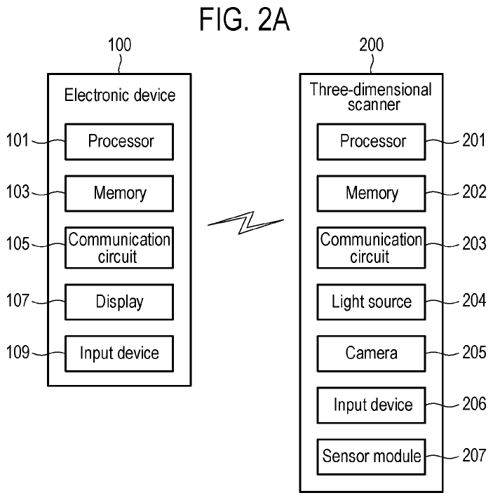

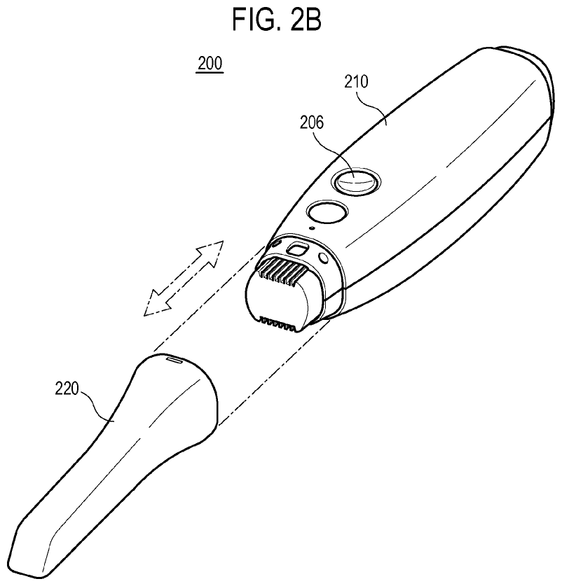

FIG. 2A is a block diagram of an electronic device 100 and a three-dimensional scanner 200 according to an embodiment of the present disclosure. The electronic device 100 and the three-dimensional scanner 200 may be communicatively connected to each other via a wired or wireless communication network, and may transmit and receive various types of data to and from each other.

The three-dimensional scanner 200 according to various embodiments may include a processor 201, a memory 202, a communication circuit 203, a light source 204, a camera 205, an input device 206, and/or a sensor module 207. At least one of the elements included in the three-dimensional scanner 200 may be omitted, or other elements may be added to the three-dimensional scanner 200. Additionally or alternatively, some elements may be integrated, or implemented as a single or multiple entities. At least some elements in the three-dimensional scanner 200 may be connected to each other via a bus, a general-purpose input/output (GPIO), a serial peripheral interface (SPI), or a mobile industry processor interface (MIPI) so as to transmit and/or receive data and/or signals.

The processor 201 of the three-dimensional scanner 200 according to various embodiments is an element capable of performing computation or data processing related to control and/or communication of the elements of the three-dimensional scanner 200, and may be operatively coupled to the elements of the three-dimensional scanner 200. The processor 201 may load instructions or data received from other elements of the three-dimensional scanner 200 into the memory 202, may process the instructions or data stored in the memory 202, and may store resulting data. According to various embodiments, the memory 202 of the three-dimensional scanner 200 may store instructions for the above-described operations of the processor 201.

According to various embodiments, the communication circuit 203 of the three-dimensional scanner 200 may establish a wired or wireless communication channel with an external device (e.g., the electronic device 100), and may transmit and receive various types of data to and from the external device. According to an embodiment, for wired communication with the external device, the communication circuit 203 may include at least one port to be connected to the external device with a wired cable. In the above case, the communication circuit 203 may communicate with the external device connected by a wired cable via the at least one port. According to an embodiment, the communication circuit 203 may be configured to include a cellular communication module so as to be connected to a cellular network (e.g., 3G, LTE, 5G, WiBro, or WiMAX). According to various embodiments, the communication circuit 203 may include a short-range communication module to transmit and receive data to and from external devices by using short-range communication (e.g., Wi-Fi, Bluetooth, Bluetooth Low Energy (BLE), UWB), but the present disclosure is not limited thereto. According to an embodiment, the communication circuit 203 may include a contactless communication module for contactless communication. The contactless communication may include at least one contactless proximity communication technology, for example, near-field communication (NFC) communication, radio frequency identification (RFID) communication, or magnetic secure transmission (MST) communication.

According to various embodiments, the light source 204 of the three-dimensional scanner 200 may emit light toward the oral cavity of the subject 20. For example, the light emitted from the light source 204 may be structured light having a predetermined pattern (e.g., a stripe pattern in which differently colored straight lines are continuously shown). The pattern of the structured light may be generated, for example, using a pattern mask or a digital micro-mirror device (DMD), but the present disclosure is not limited thereto. The camera 205 of the three-dimensional scanner 200 according to various embodiments may acquire an image of the oral cavity of the subject 20 by receiving light reflected by the oral cavity of the subject 20. The camera 205 may include a left camera corresponding to the left field of view and a right camera corresponding to the right field of view, for example, in order to construct a three-dimensional image by using optical triangulation. The camera 205 may include at least one image sensor, such as a CCD sensor or a CMOS sensor.

The input device 206 of the three-dimensional scanner 200 according to various embodiments may receive a user input for controlling the three-dimensional scanner 200. The input device 206 may include buttons for receiving push manipulation from the user 10, a touch panel for detecting touch from the user 10, and a speech recognition device including a microphone. For example, the user 10 may use the input device 206 to control the start or stop of scanning.

The sensor module 207 of the three-dimensional scanner 200 according to various embodiments may detect an operational state of the three-dimensional scanner 200 or an external environmental state (e.g., a user's motion) and generate an electrical signal corresponding to the detected state. The sensor module 207 may include, for example, at least one of a gyro sensor, an accelerometer, a gesture sensor, a proximity sensor, or an infrared sensor. The user 10 may use the sensor module 207 to control the start or stop of scanning. For example, when the user 10 is moving while holding the three-dimensional scanner 200 in hand, the three-dimensional scanner 200 may be controlled to start a scanning operation of the processor 201 when an angular velocity measured by the sensor module 207 exceeds a predetermined threshold.

According to an embodiment, the three-dimensional scanner 200 may start scanning by receiving a user input for starting scanning through the input device 206 of the three-dimensional scanner 200 or an input device 206 of the electronic device 100, or in response to processing in a processor 201 of the three-dimensional scanner 200 or the processor 201 of the electronic device 100. When the user 10 scans the interior of the oral cavity of the subject 20 by using the three-dimensional scanner 200, the three-dimensional scanner 200 may generate a two-dimensional image of the oral cavity of the subject 20, and may transmit the two-dimensional image of the oral cavity of the subject 20 to the electronic device 100 in real time. The electronic device 100 may display the received two-dimensional image of the oral cavity of the subject 20 on a display. Further, the electronic device 100 may generate (construct) a three-dimensional image of the oral cavity of the subject 20, based on the two-dimensional image of the oral cavity of the subject 20, and may display the three-dimensional image of the oral cavity on the display. The electronic device 100 may display the three-dimensional image, which is being generated, on the display in real time.

The electronic device 100 according to various embodiments may include at least one processor 101, at least one memory 103, a communication circuit 105, a display 107, and/or an input device 109. At least one of the elements included in the electronic device 100 may be omitted, or other elements may be added to the electronic device 100. Additionally or alternatively, some elements may be integrated, or implemented as a single or multiple entities. At least some elements in the electronic device 100 may be connected to each other via a bus, a general-purpose input/output (GPIO), a serial peripheral interface (SPI), or a mobile industry processor interface (MIPI) so as to exchange data and/or signals.

According to various embodiments, the at least one processor 101 of the electronic device 100 may be an element capable of performing computation or data processing related to control and/or communication of the elements of the electronic device 100 (e.g., memory 103). The at least one processor 101 may be operatively coupled to the elements of the electronic device 100, for example. The at least one processor 101 may load instructions or data received from other elements of the electronic device 100 into the at least one memory 103, may process the instructions or data stored in the at least one memory 103, and store the resulting data.

According to various embodiments, the at least one memory 103 of the electronic device 100 may store instructions for operations of the at least one processor 101. The at least one memory 103 may store correlation models constructed based on a machine learning algorithm. The at least one memory 103 may store data (e.g., a two-dimensional image of the oral cavity acquired through oral scanning) received from the three-dimensional scanner 200.

According to various embodiments, the communication circuit 105 of the electronic device 100 may establish a wired or wireless communication channel with an external device (e.g., the three-dimensional scanner 200 or the cloud server), and may transmit or receive various types of data to or from the external device. According to an embodiment, for wired communication with the external device, the communication circuit 105 may include at least one port so as to be connected to the external device through a wired cable. In the above case, the communication circuit 105 may communicate with the external device connected through the wired cable via the at least one port. According to an embodiment, the communication circuit 105 may be configured to include a cellular communication module so as to be connected to a cellular network (e.g., 3G, LTE, 5G, WiBro, or WiMAX). According to various embodiments, the communication circuit 105 may include a short-range communication module to transmit and receive data to and from external devices by using short-range communication (e.g., Wi-Fi, Bluetooth, Bluetooth Low Energy (BLE), or UWB), but the present disclosure is not limited thereto. According to an embodiment, the communication circuit 105 may include a contactless communication module for contactless communication. The contactless communication may include at least one contactless proximity communication technology such as, for example, near-field communication (NFC) communication, radio frequency identification (RFID) communication, or magnetic secure transmission (MST) communication.

The display 107 of the electronic device 100 according to various embodiments may display various screens based on control of the processor 101. The processor 101 may display, on the display 107, a two-dimensional image of the oral cavity of subject 20 received from the three-dimensional scanner 200 and/or a three-dimensional image of the oral cavity in which the inner structure of the oral cavity is modeled. For example, the two-dimensional image and/or the three-dimensional image of the oral cavity may be displayed through a particular application. In the above case, the user 10 may edit, save, and delete the two-dimensional image and/or the three-dimensional image of the oral cavity.

The input device 109 of the electronic device 100 according to various embodiments may receive instructions or data, which are to be used by an element (e.g., the at least one processor 101) of the electronic device 100, from an external source (e.g., a user) of the electronic device 100. The input device 109 may include, for example, a microphone, a mouse, or a keyboard. According to an embodiment, the input device 109 may be implemented in the form of a touch sensor panel that may be coupled to the display 107 to recognize contact or proximity of various external objects.

FIG. 2B is a perspective diagram of a three-dimensional scanner 200 according to an embodiment of the present disclosure. The three-dimensional scanner 200 according to various embodiments may include a body 210 and a probe tip 220. The body 210 of the three-dimensional scanner 200 may be formed in a shape that is easy for the user 10 to grip and use by hand. The probe tip 220 may be shaped for easy insertion into and removal from the oral cavity of the subject 20. Further, the body 210 may be coupled to and detachable from the probe tip 220. Inside the body 210, the elements of the three-dimensional scanner 200 illustrated in FIG. 2A may be disposed. An opening may be formed at one end of the body 210 so that light output from the light source 204 can be emitted onto the subject 20 through the opening. Light emitted through the opening may be reflected by the subject 20 and introduced again through the opening. The reflected light introduced through the opening may be captured by the camera to generate an image of the subject 20. The user 10 may start scanning by using the input device 206 (e.g., a button) of the three-dimensional scanner 200. For example, when the user 10 touches or presses the input device 206, light from the light source 204 may be emitted onto the subject 20.

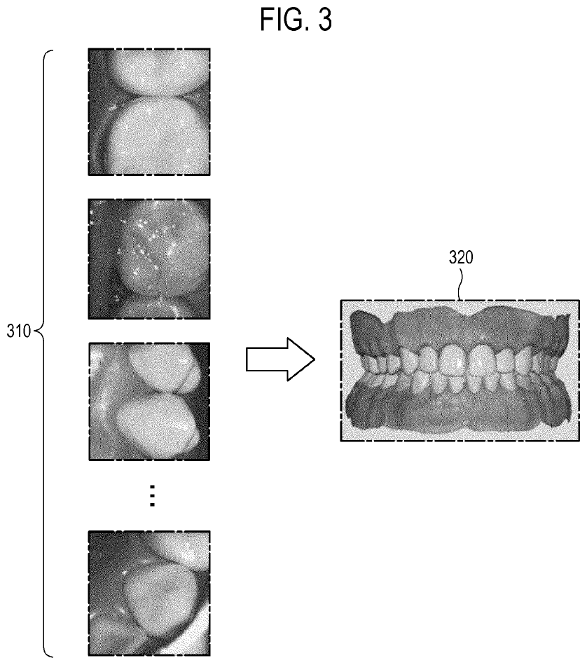

FIG. 3 illustrates a method for generating a three-dimensional image 320 of an oral cavity according to an embodiment of the present disclosure. In the present disclosure, when a “three-dimensional scan data set” is visually represented, the “three-dimensional scan data set” may be referred to as a “three-dimensional image.” The electronic device 100 according to an embodiment of the present disclosure may acquire the at least one two-dimensional scan image by scanning with the three-dimensional scanner 200, and generate a three-dimensional scan data set regarding the surface of the subject 20 based on the at least one acquired two-dimensional scan image. The three-dimensional scan data set may include multiple three-dimensional coordinate values.

In an embodiment, the user 10 may move the three-dimensional scanner 200 to scan the interior of the oral cavity of the subject 20, in which case the three-dimensional scanner 200 may acquire at least one two-dimensional scan image 310 of the oral cavity of the subject 20. For example, the three-dimensional scanner 200 may acquire a two-dimensional scan image of an area containing the incisors of the subject 20, a two-dimensional scan image of an area containing the molars of the subject 20, and so forth. The three-dimensional scanner 200 may transmit the at least one acquired two-dimensional scan image 310 to the electronic device 100.

In another embodiment, the user 10 may move the three-dimensional scanner 200 to scan a diagnostic model of the oral cavity, and to acquire at least one two-dimensional scan image of the diagnostic model of the oral cavity. Hereinafter, for ease of description, the description will be made assuming that an image of the oral cavity of the subject 20 is acquired by scanning the interior of the oral cavity of the subject 20, but the present disclosure is not limited thereto.

The electronic device 100 according to various embodiments may convert each of the at least one two-dimensional scan image 310 of the oral cavity of the subject 20 into a set of multiple points having three-dimensional coordinate values. For example, the electronic device 100 may convert each of the at least one two-dimensional scan image 310 into a point cloud set which is a set of data points having three-dimensional coordinate values. In the present disclosure, the term “point cloud set,” which is a set of data points having three-dimensional coordinate values, may be used interchangeably with “three-dimensional scan data set.” The three-dimensional scan data set, which includes three-dimensional coordinate values generated based on the at least one two-dimensional scan image 310, may be stored as raw data regarding the oral cavity of the subject 20. In one example, the electronic device 100 may align the three-dimensional scan data set, which is a set of data points having three-dimensional coordinate values, to generate a three-dimensional scan data set that includes fewer data points. In another example, the electronic device 100 may reconfigure (reconstruct) the three-dimensional scan data set regarding the oral cavity. For example, the electronic device 100 may reconfigure the multiple data points by merging at least some of the data in the three-dimensional scan data set stored as raw data, using a Poisson algorithm, and transform the data points into a closed three-dimensional surface. As a result, the electronic device 100 may reconfigure a three-dimensional scan data set of the oral cavity of the subject 20.

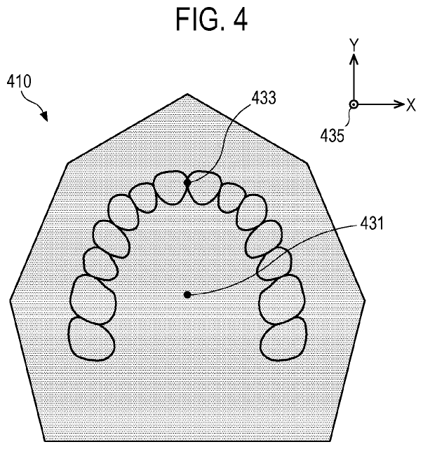

FIG. 4 illustrates exemplary plane data corresponding to a virtual occlusal plane according to an embodiment of the present disclosure. In general, occlusion refers to a state of engagement of the teeth, and refers to a mutual positioning of the upper and lower teeth when the maxilla and the mandible are closed. In this context, an occlusal plane refers to a surface where the maxilla and the mandible face each other, or the upper and lower teeth face each other. In the present disclosure, a virtual occlusal plane may refer to a virtual plane used to represent the occlusal surface of a tooth as a flat surface.

The electronic device 100 according to an embodiment of the present disclosure may generate first plane data corresponding to a virtual occlusal plane 410. The first plane data may include a center point 431 and a normal vector 435 for determining one plane. For example, the normal vector 435 of the first plane data may be a vector perpendicular to the virtual occlusal plane and parallel to the z-axis in a three-dimensional Cartesian coordinate system. The first plane data may further include an anterior tooth point 433 corresponding to anterior teeth among the teeth that are virtually present on the virtual occlusal plane. The anterior tooth point 433 may be, for example, a center point of two incisors included in the anterior teeth among the teeth present on the virtual occlusal plane. In the present disclosure, the center point 431, the anterior tooth point 433, and the normal vector 435 in the first plane data may be referred to as a first center point, a first anterior tooth point, and a first normal vector, respectively.

The electronic device 100 according to an embodiment of the present disclosure may generate the first plane data corresponding to the virtual occlusal plane 410 based on a signal input from a user via the input device 109. The user may input at least one among the first center point, the first normal vector, and the first anterior tooth point for determining the first plane data via the input device 109 of the electronic device 100, and the electronic device 100 may generate the first plane data based on the input value. In addition, the electronic device 100 may generate the first plane data by using a predetermined default value for at least one among the first center point, the first normal vector, and the first anterior tooth point.

The electronic device 100 according to an embodiment of the present disclosure may determine multiple reference coordinate values based on an acquired three-dimensional scan data set. For example, when a three-dimensional scan data set of the oral cavity of the subject 20 includes a scan data set corresponding to the maxilla and a scan data set corresponding to the mandible, the electronic device 100 may determine multiple reference coordinate values based on one of the scan data set corresponding to the maxilla and the scan data set corresponding to the mandible. In the present disclosure, the electronic device 100 may generate plane data corresponding to the occlusal plane of the oral cavity of the subject 20 based on the multiple reference coordinate values.

The electronic device 100 according to an embodiment of the present disclosure may display a three-dimensional scan data set to a user and determine multiple reference coordinate values based on a signal input from the user via the input device 109. In the present disclosure, the reference coordinate values may be values that serve as a basis for determining the occlusal plane of the oral cavity of the subject 20 in the three-dimensional coordinate space in which the three-dimensional scan data set regarding the oral cavity of the subject is represented. Since one unique plane is generally determined when three different points that are not located in a straight line in the three-dimensional coordinate space are determined, the electronic device 100 according to the present disclosure may determine multiple reference coordinate values by receiving, from the user, an input of the locations of at least three points (that are not located in a straight line) included in the three-dimensional scan data set. The electronic device 100 according to the present disclosure may recalculate three points for determining an occlusal plane through a predetermined computation when receiving an input of locations of more than three points from the user. As an example of determining the multiple reference coordinate values based on the user's input, the electronic device 100 may generate (construct) a three-dimensional image of the oral cavity of the subject 20 by using the three-dimensional scanner 200, and then display the three-dimensional image of the oral cavity on the display 107. In this case, the electronic device 100 may receive information about three or more different points on the three-dimensional image from the user via the input device 109. For example, the input device 109 may be a mouse, a touch pen, or a touch pad, and the user may select three or more different points by clicking or touching any points within the three-dimensional image displayed via the display 107, and, as a result, the electronic device 100 may determine multiple reference coordinate values for determining an occlusal plane.

The electronic device 100 according to an embodiment of the present disclosure may determine multiple reference coordinate values from the three-dimensional scan data set by using a trained artificial neural network model. Data regarding the trained artificial neural network model (e.g., weights or bias values of the model) may be stored in the memory 103 of the electronic device 100 according to the present disclosure. The electronic device 100 may input curvature information of a two-dimensional scan image into the trained artificial neural network model to identify a tooth number of the two-dimensional scan image, and may determine multiple reference coordinate values from a three-dimensional scan data set based on the identification. Specifically, the electronic device 100 may obtain curvature information of each of at least one two-dimensional scan image 310 acquired by the three-dimensional scanner 200, and may identify a tooth number of the corresponding two-dimensional scan image by inputting the obtained curvature information into the trained artificial neural network model. The electronic device 100 may set the identified tooth number of the two-dimensional scan image as a tooth number of a three-dimensional coordinate value corresponding to the two-dimensional scan image. Hereinafter, a method by which the electronic device 100 of the present disclosure identifies a tooth number of each two-dimensional scan image will be first described.

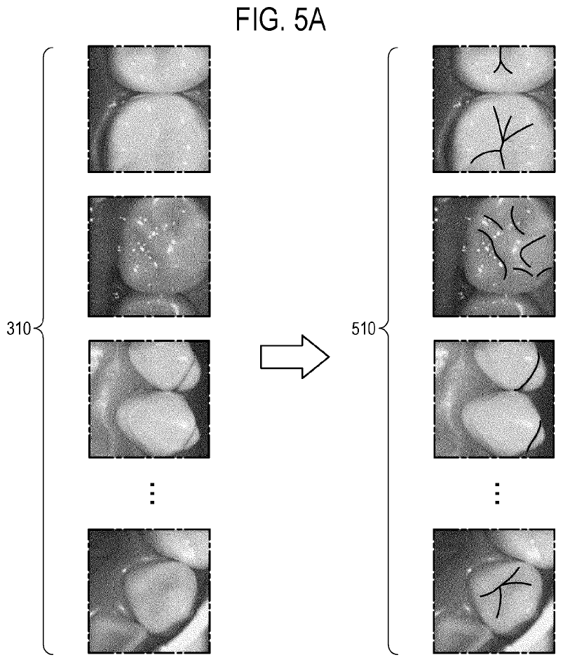

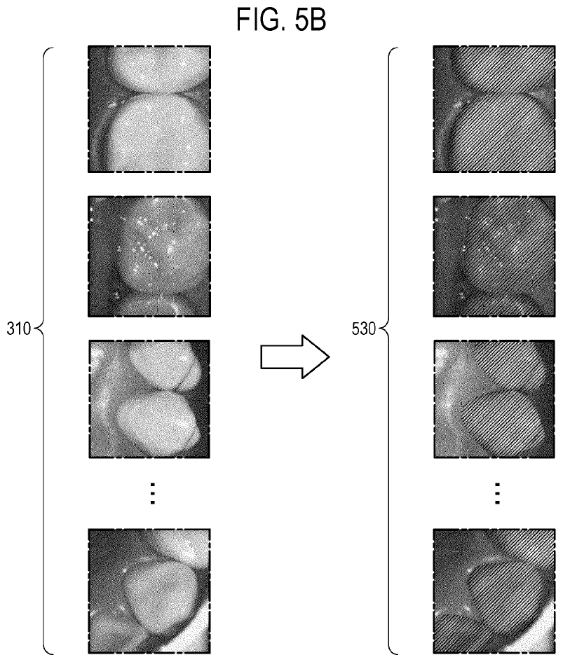

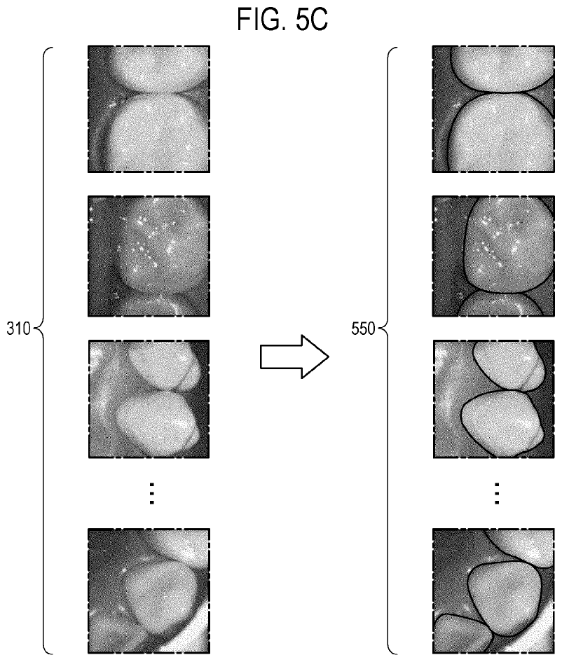

FIG. 5A is an illustrative diagram visually depicting curvature information of each of at least one two-dimensional scan image according to an embodiment of the present disclosure. In the present disclosure, curvature information refers to quantitative information for representing a degree of curvature of a tooth surface, and may vary depending on the type of tooth. That is, curvature information of molars and curvature information of canines may be distinguished from each other, and even in the same category, such as molars, a molar corresponding to a tooth 18 and a molar corresponding to a tooth 17 may have different pieces of curvature information. In the present disclosure, the curvature information may include a coordinate value of at least one point, among multiple points in a two-dimensional image, which is determined according to predetermined criteria for representing a curvature of a tooth surface. The curvature information may be information determined based on an elevation (i.e., height) of the multiple points included in a tooth area in the two-dimensional image. The curvature information may be generated from a tooth surface in the two-dimensional scan image so as to be the same as or similar to, for example, contour lines on a two-dimensional map.

Curvature information according to an embodiment of the present disclosure may be generated by the electronic device 100 based on a distance between the light source 204 of the three-dimensional scanner 200 and the tooth surface during the process of acquiring the at least one two-dimensional scan image 310 via the three-dimensional scanner 200. Curvature information according to another embodiment of the present disclosure may also be calculated, by the electronic device 100, using the contrast of tooth areas included in the two-dimensional image obtained via the three-dimensional scanner 200. For example, when the curvature information is obtained using the contrast of the tooth areas included in the two-dimensional image, it may be determined that points having relatively lower brightness than surrounding points are points to be included in the curvature information. In addition, the curvature information may be calculated by an external device with respect to a specific two-dimensional scan image, and then may be received via the communication circuit 105 of the electronic device 100 and stored in the memory 103 of the electronic device 100. The foregoing description of a method for calculating curvature information or a subject of calculating curvature information is exemplary and does not limit the present disclosure

In an embodiment of the present disclosure, multiple pieces of curvature information 510 generated for the at least one two-dimensional scan image 310 may be shown as information representing areas of tooth surfaces that are lower in height than surrounding areas when illustrated in a two-dimensional image format, as in FIG. 5A. Here, the criterion of “lower height” may be established based on the relative position between the light source 204 of the three-dimensional scanner 200 and the tooth surfaces at the time of acquiring the two-dimensional image. The above description of curvature information, as made with reference to FIG. 5A, is merely an illustrative example and does not limit the present disclosure. Therefore, any type of information indicating the degree of curvature of a tooth surface may be included in the curvature information of the present disclosure.

An artificial neural network model according to the present disclosure may receive curvature information for a two-dimensional training image so as to be trained to predict a tooth number of the two-dimensional training image. In the present disclosure, the “two-dimensional training image” is a term used to represent training data for the artificial neural network model, and may be an image acquired by scanning of the three-dimensional scanner 200, or may be an image transmitted from an external device for training.

An artificial neural network model according to an embodiment of the present disclosure may be trained based on a training data set that includes curvature information of each of at least one two-dimensional training image and a tooth number corresponding to each of the at least one two-dimensional training image. That is, the training data set may include multiple pieces of training data, and each piece of training data may be data that includes curvature information for a specific two-dimensional training image and a tooth number of the two-dimensional training image. The artificial neural network model according to the present disclosure may be trained by setting curvature information of a two-dimensional training image as input data and the tooth number of the two-dimensional training image, which corresponds to the input curvature information, as output data. Hereinafter, for convenience of description, the artificial neural network model will be described as being trained by the electronic device 100 of the present disclosure. However, the present disclosure is not limited thereto, and the artificial neural network model may be completely trained by an external device, and may then be transferred from the external device to the electronic device 100 and used. The electronic device 100 according to the present disclosure may obtain the above-described curvature information for each two-dimensional training image, and then input the obtained curvature information into the artificial neural network model. The electronic device 100 may train, based on the input curvature information, the artificial neural network model to predict a tooth number of a tooth included in the two-dimensional training image.

The electronic device 100 according to an embodiment of the present disclosure may input curvature information of each of at least one two-dimensional scan image to the trained artificial neural network model, and identify a tooth number of each of the at least one two-dimensional scan image based on an output of the artificial neural network model performing a computation based on the input curvature information. For example, the electronic device 100 may input curvature information of a two-dimensional image including a tooth 11 (i.e., a left incisor) into the trained artificial neural network model to identify that the tooth included in the two-dimensional image has a tooth number of 11.

In an embodiment of the present disclosure, a training data set for training the artificial neural network model may include curvature information of each of the at least one two-dimensional training image and a tooth number corresponding to each of the at least one two-dimensional training image, but may further include at least one selected from the group of size information of each of the at least one two-dimensional training image and shape information of each of the at least one two-dimensional training image. In other words, a training data set may include multiple pieces of training data, and each piece of the training data may be data that includes curvature information of a specific two-dimensional training image, a tooth number corresponding to that two-dimensional training image, and further includes at least one selected from the group of size information and shape information of the two-dimensional training image.

The artificial neural network model according to an embodiment of the present disclosure may be trained based on a training data set that further includes, in the training data set including the curvature information and the tooth number, at least one selected from the group of size information of each of the at least one two-dimensional training image and shape information of each of the at least one two-dimensional training image. The artificial neural network model may be trained to output a tooth number of a two-dimensional training image by using, as input data, data that further includes, in addition to curvature information of a two-dimensional training image, at least one selected from the group of size information and shape information of the two-dimensional training image.

FIG. 5B is an illustrative diagram visually depicting size information of each of at least one two-dimensional scan image according to an embodiment of the present disclosure. In the present disclosure, size information may be quantitative information for representing the size of a tooth. The size information may be information that is determined based on the size of a tooth area in a two-dimensional image. The electronic device 100 according to the present disclosure may identify an area corresponding to a tooth in each of the at least one two-dimensional scan image 310, and may calculate multiple pieces of size information based on the size of the identified tooth area. The size information according to the present disclosure may have a predetermined real value. For example, size information of a molar portion may have a real value of “3,” and size information of an incisor portion may have a real value of “1.” Here, the real value of the size information is a value for relative comparison, and thus may have a magnitude of a predetermined value. To match measurement criteria (or scale) of size information acquired from at least one two-dimensional scan image 310, the electronic device 100 may correct the size of the two-dimensional scan image based on a calculated distance between the light source 204 of the three-dimensional scanner 200 and a tooth surface. In addition, to match measurement criteria (or scale) of size information acquired from the at least one two-dimensional scan image 310, the electronic device 100 may calculate, in the step of acquiring the two-dimensional scan image, size information by using the two-dimensional scan image only when the light source 204 of the three-dimensional scanner 200 and the tooth surface are separated from each other by a predetermined distance. In accordance with an embodiment of the present disclosure, when a tooth area on which the electronic device 100 is based for obtaining size information from each of the at least one two-dimensional scan image 310 is represented as a two-dimensional image, the tooth area may be represented as shown by reference numeral 530 in FIG. 5B. The above description of the size information, as made with reference to FIG. 5B, is merely an illustrative example and does not limit the present disclosure.

FIG. 5C is an illustrative diagram visually depicting shape information of each of at least one two-dimensional scan image according to an embodiment of the present disclosure. Shape information according to the present disclosure may be quantitative information for representing contours of a tooth. The shape information may be information that is determined based on multiple points that form a border of a tooth area in a two-dimensional image. The electronic device 100 according to the present disclosure may identify an area corresponding to a tooth in each of at least one two-dimensional scan image 310, and may calculate shape information 550 based on coordinates of multiple points included in the edge of the identified tooth area. For example, the shape information 550 generated for each of the at least one two-dimensional scan image 310, when represented as a two-dimensional image as shown in FIG. 5C, may include coordinates of multiple points corresponding to a boundary distinguishing between a tooth area and a gum area. The above description of the shape information, as made with reference to FIG. 5C, is merely an illustrative example and does not limit the present disclosure.

When an artificial neural network model according to an embodiment of the present disclosure is trained based on a training data set that further includes at least one selected from the group of size information and shape information in addition to curvature information, the electronic device 100 may input at least one selected from the group of the size information and the shape information, along with curvature information of each of the at least one two-dimensional scan image, into the trained artificial neural network model, and may identify a tooth number corresponding to each of the at least one two-dimensional scan image, based on an output of the artificial neural network model which performs a computation based on the input curvature information and performs a computation additionally based on at least one selected from the group of the input size information and the input shape information.

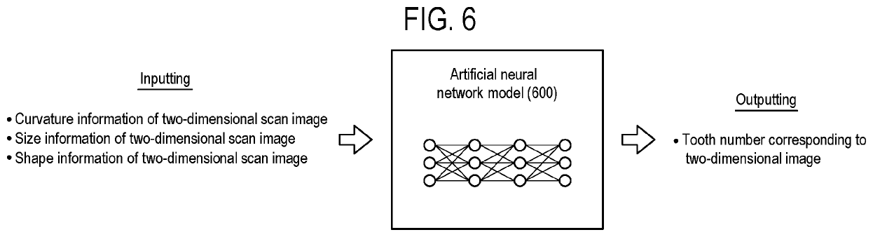

FIG. 6 conceptually illustrates a method for using an artificial neural network model according to an embodiment of the present disclosure. An artificial neural network model 600 of the present disclosure may receive curvature information of a two-dimensional scan image alone to identify a tooth number of the two-dimensional scan image. Further, the artificial neural network model 600 may additionally receive, in addition to curvature information of a two-dimensional scan image, at least one selected from the group of size information and shape information of the two-dimensional scan image to identify a tooth number of the two-dimensional scan image.

In another embodiment of the present disclosure, although curvature information of a two-dimensional scan image including a specific tooth X has been input into the trained artificial neural network model 600, when the output confidence score of the artificial neural network model for the two-dimensional scan image is lower than a predetermined threshold, the electronic device 100 may input the curvature information and size information of the two-dimensional scan image including the tooth X into the artificial neural network model 600 to acquire a tooth number of the two-dimensional scan image. At this time, the electronic device 100 may additionally input shape information rather than the size information, or may input both the size information and the shape information together with the curvature information. In an embodiment of the present disclosure, an artificial neural network model trained on the basis of a training data set that further includes at least one selected from the group of size information and shape information in addition to curvature information of a two-dimensional image may identify (predict) a tooth number of the two-dimensional scan image based on the additional information in addition to the curvature information, thereby having the effect of further improving the accuracy of identification of the tooth number.

The electronic device 100 according to an embodiment of the present disclosure may determine, based on a trained artificial neural network model, tooth numbers of three-dimensional coordinate values corresponding to two-dimensional scan images by using a tooth number identified for each of the two-dimensional scan images. The three-dimensional coordinate values corresponding to the two-dimensional scan images may be data included in a three-dimensional scan data set. As described above, the electronic device 100 of the present disclosure may generate a three-dimensional scan data set by converting each of the at least one two-dimensional scan image 310 into a point cloud set, which is a set of data points having three-dimensional coordinate values. In this case, the electronic device 100 according to an embodiment of the present disclosure may, when calculating at least one three-dimensional coordinate value for generating the three-dimensional scan data set from each two-dimensional scan image, determine a tooth number identified from the two-dimensional image as a tooth number of the corresponding three-dimensional coordinate value. For example, it is assumed that the three-dimensional scanner 200 acquires a two-dimensional scan image of a tooth 27 during a scan of the oral cavity and generates a three-dimensional scan data set regarding the tooth 27 having at least one three-dimensional coordinate value. In this case, the electronic device 100 may input curvature information of the two-dimensional scan image including the tooth 27 into the trained artificial neural network model 600. The curvature information may be calculated by the electronic device 100. Based on the output of the artificial neural network model 600, the electronic device 100 may identify a tooth number (i.e., no. 27) of the two-dimensional scan image. As a result, the electronic device 100 may determine that the tooth number of the three-dimensional coordinate value generated from the two-dimensional scan image including the tooth number 27 is 27. Thus, the electronic device 100 of the present disclosure may convert a two-dimensional scan image into a set of data points having three-dimensional coordinate values, and may further identify a tooth number of the two-dimensional scan image based on the trained artificial neural network model 600, and may thus determine a tooth number of a finally generated three-dimensional coordinate value.

After determining the tooth number of the three-dimensional coordinate value based on the trained artificial neural network model, the electronic device 100 according to an embodiment of the present disclosure may determine multiple reference coordinate values from the three-dimensional scan data set. For example, when the electronic device 100 determines a tooth number of a three-dimensional coordinate value included in the three-dimensional scan data set based on a two-dimensional scan image, each tooth may include multiple three-dimensional coordinate values, so that the multiple three-dimensional coordinate values may have the same tooth number. That is, the three-dimensional coordinate values and the tooth number generated from the two-dimensional image have a many-to-one data relationship, so that the multiple three-dimensional coordinate values may have the same tooth number. For example, when a three-dimensional scan data set includes 200 three-dimensional coordinate values in an area corresponding to a tooth 48 (the lower left molar), all of the three-dimensional coordinate values may have a tooth number of 48.

The electronic device 100 according to an embodiment of the present disclosure may determine a representative coordinate value of a corresponding tooth based on the multiple three-dimensional coordinate values determined to have the same tooth number, and may determine multiple reference coordinate values based on the determined representative coordinate value. In the present disclosure, the “representative coordinate value” is a value intended to be representative of multiple three-dimensional coordinate values determined to have the same tooth number, and may be distinguished from a “reference coordinate value” which, in the present disclosure, is the basis for generating plane data corresponding to an occlusal plane regardless of a tooth number. The electronic device 100 according to an embodiment of the present disclosure may determine, based on various methods, a representative coordinate value of the corresponding tooth from the multiple three-dimensional coordinate values having the same tooth number.

The electronic device 100 according to an embodiment of the present disclosure may determine a center point of the multiple three-dimensional coordinate values as the representative coordinate value of the corresponding tooth. For example, it is assumed that a set of multiple three-dimensional coordinate values corresponding to a tooth N includes (7.8, 9.5, 6.8), (3.4, 9.4, 7.1), and (9.0, 8.5, 6.8). In this case, the representative coordinate value of the tooth N may be determined to be (6.73, 9.13, 6.9), which is the center point of the coordinate values included in the set.

The electronic device 100 according to another embodiment of the present disclosure may determine that when each ordered pair of multiple three-dimensional coordinate values is expressed as (X, Y, Z), a three-dimensional coordinate value having the (X, Y) value closest to a midpoint of multiple (X, Y) ordered pairs having only the first value and the second value is a representative coordinate value of the corresponding tooth. For example, it is assumed that a set of multiple three-dimensional coordinate values corresponding to the tooth N includes (7.8, 9.5, 6.8), (3.4, 9.4, 7.1), and (9.0, 8.5, 6.8). In this case, a midpoint of (X, Y) coordinate values (i.e., (7.8, 9.5), (3.4, 9.4), and (9.0, 8.5)) is (6.73, 9.13). Further, according to the Euclidean distance calculation, the distance between (7.8, 9.5) and the midpoint is about 1.13, the distance between (3.4, 9.4) and the midpoint is about 3.34, and the distance between (9.0, 8.5) and the midpoint is about 2.35, so a three-dimensional coordinate value having X and Y values closest to (6.73, 9.13) may be (7.8, 9.5, 6.8). Therefore, the representative coordinate value of the tooth N may be determined to be (7.8, 9.5, 6.8). When determining a representative coordinate value of a corresponding tooth from multiple three-dimensional coordinate values based on the above-described embodiment, there is an effect that the most central data point on the occlusal plane of the tooth (i.e., the X-Y plane) among multiple data points constituting the tooth from the perspective of viewing the tooth in a direction perpendicular to the occlusal plane of the tooth may be determined to be the representative coordinate value.

In another embodiment, when an ordered pair of each of multiple three-dimensional coordinate values is represented by (X, Y, Z), the electronic device 100 may determine that a three-dimensional coordinate value having the largest Z value, among the multiple three-dimensional coordinate values, is the representative coordinate value of a corresponding tooth. The foregoing description of a method for determining a representative coordinate value of a tooth is merely for illustrative purposes and does not limit the present disclosure.

The electronic device 100 according to an embodiment of the present disclosure may determine multiple reference coordinate values from a representative coordinate value of each tooth. The electronic device 100 may set the representative coordinate value of the corresponding tooth as a reference coordinate value. For example, the electronic device 100 may determine representative coordinate values of the left third central incisor, the right third incisor, the left canine, and the right canine as the respective reference coordinate values thereof. The electronic device 100 may calculate reference coordinate values from representative coordinate values of two or more teeth. For example, the electronic device 100 may determine a reference coordinate value corresponding to an anterior tooth point by calculating a midpoint of representative coordinate values of two anterior teeth. In another example, the electronic device 100 may calculate a midpoint of representative coordinate values of teeth 16 to 18 to determine reference coordinate values corresponding to the left molars.

The electronic device 100 according to an embodiment of the present disclosure may determine multiple reference coordinate values based on a tooth number determined for each of the multiple three-dimensional coordinate values. For example, the electronic device 100 may calculate a reference coordinate value corresponding to an anterior tooth point by calculating a midpoint of multiple three-dimensional coordinate values determined to have a tooth number of 11 and multiple three-dimensional coordinate values determined to have a tooth number of 21. In another example, the electronic device 100 may calculate a midpoint of multiple three-dimensional coordinate values having tooth numbers of 16 to 18 to calculate reference coordinate values corresponding to the left molars.

The foregoing description of a method for determining a reference coordinate value based on a tooth number determined for each of multiple three-dimensional coordinate values is merely for illustrative purposes and does not limit the present disclosure. That is, the electronic device 100 of the present disclosure may determine a representative coordinate value of each tooth and determine multiple reference coordinate values from the determined representative coordinate value, or may determine multiple reference coordinate values from a tooth number determined for each of the multiple three-dimensional coordinate values without determining a representative coordinate value of each tooth.

The electronic device 100 according to an embodiment of the present disclosure may generate, based on the multiple reference coordinate values, second plane data corresponding to the occlusal plane of the oral cavity of a subject. In the present disclosure, the second plane data may include a second center point, a second anterior tooth point, and a second normal vector. The second plane data, which is generated based on a three-dimensional scan data set regarding the subject's oral cavity, may be distinguished from first plane data corresponding to a virtual occlusal plane.

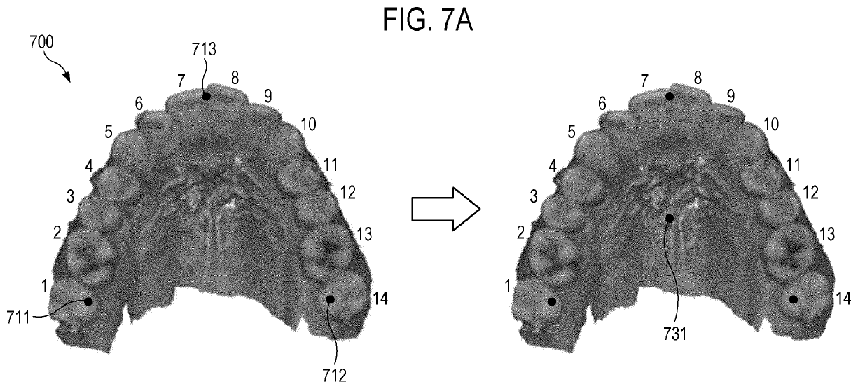

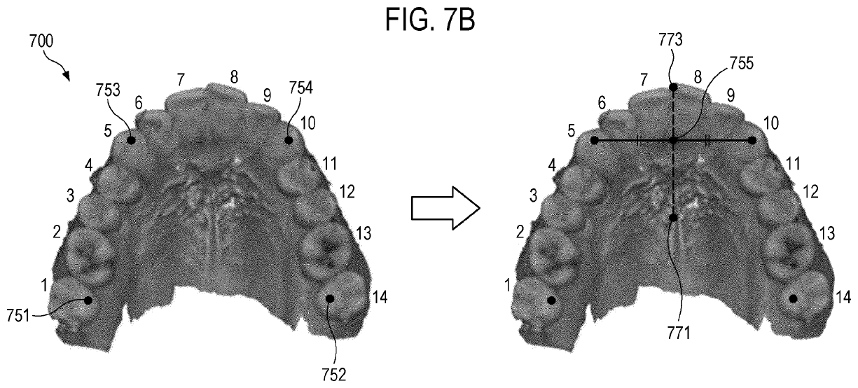

The electronic device 100 according to an embodiment of the present disclosure may generate the second plane data representing the occlusal plane of the subject's oral cavity based on various predetermined calculation methods. The electronic device 100 may determine whether the multiple reference coordinate values include a first coordinate value included in a left molar area, a second coordinate value included in a right molar area, and a third coordinate value included in an anterior tooth area. The electronic device 100 may store the third coordinate value as an anterior tooth point in the second plane data. The electronic device 100 may calculate a center point of the first coordinate value, the second coordinate value, and the third coordinate value as a center point of the second plane data. The electronic device 100 may calculate, as a normal vector of the second plane data, a vector perpendicular to a plane including the first coordinate value, the second coordinate value, and the third coordinate value. This will be described in more detail below with reference to FIG. 7A.

FIG. 7A illustrates a method by which the electronic device 100 according to an embodiment of the present disclosure generates plane data from a three-dimensional scan data set. In FIG. 7A, for ease of description, a method for generating plane data based on a data set corresponding to the maxilla of a subject is illustrated. However, the present disclosure is not limited thereto, and plane data may be generated based on a data set corresponding to the mandible of the subject, or plane data may be generated based on all of the data sets corresponding to the maxilla and the mandible of the subject. The electronic device 100 may identify a first coordinate value 711, which is included in a left molar area, among multiple reference coordinate values determined based on a three-dimensional scan data set 700. The electronic device 100 may identify a second coordinate value 712, which is included in a right molar area, among the multiple reference coordinate values determined based on the three-dimensional scan data set 700. The electronic device 100 may identify a third coordinate value 713, which is included in an anterior tooth area, among the multiple reference coordinate values determined based on the three-dimensional scan data set 700. The electronic device 100 may store the third coordinate value 713 such that the anterior tooth point in the second plane data is the third coordinate value 713, that is, may store the third coordinate value 713 as the anterior tooth point in the second plane data. The electronic device 100 may calculate a center point of the first coordinate value 711, the second coordinate value 712, and the third coordinate value 713 as a center point 731 in the second plane data. The electronic device 100 may store the center point of the first coordinate value 711, the second coordinate value 712, and the third coordinate value 713 as the center point 731 in the second plane data. The electronic device 100 may produce a plane determined by the first coordinate value 711, the second coordinate value 712, and the third coordinate value 713, and calculate a vector perpendicular to the plane, thereby calculating a normal vector of the second plane data. Thus, the electronic device 100 of the present disclosure may generate the second plane data, which represents the occlusal plane of the subject's oral cavity, from the multiple reference coordinate values.

The electronic device 100 according to an embodiment of the present disclosure may align a three-dimensional scan data set on a virtual occlusal plane by matching the second plane data with first plane data. By matching the first plane data representing the virtual occlusal plane 410 with the second plane data representing the occlusal plane of the subject's oral cavity, the electronic device 100 may align a three-dimensional scan data set of the subject on the virtual occlusal plane 410. The electronic device 100 may perform a predetermined computation on the second plane data representing the occlusal surface of the subject's oral data to match the first plane data and second plane data such that the second plane data matches the first plane data representing the virtual occlusal surface 410. The electronic device 100 may perform transformations, for example, a translation transformation, a rotation transformation, or the like, with respect to the second plane data.

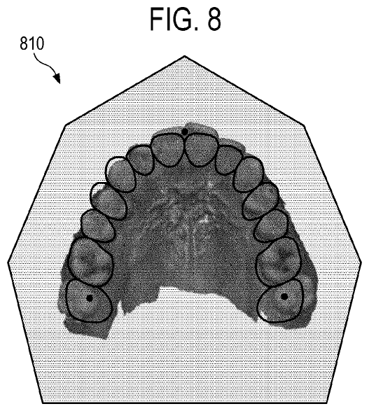

FIG. 8 is an illustrative diagram depicting a result of matching plane data corresponding to a virtual occlusal plane to plane data corresponding to the occlusal plane of a subject's oral cavity by the electronic device 100 according to an embodiment of the present disclosure. For convenience of description, plane data of a virtual occlusal plane is referred to as first plane data, and plane data corresponding to the occlusal plane of a subject's oral cavity is referred to as second plane data. The electronic device 100 may match a first center point included in the first plane data and a second center point included in the second plane data. That is, the electronic device 100 may match the center points of the first plane data and the second plane data with each other. The electronic device 100 may match a first normal vector included in the first plane data and a second normal vector included in the second plane data. By matching the normal vectors of the first plane data and the second plane data, the virtual occlusal plane and the occlusal plane of the subject's oral cavity may have a parallel positional relationship therebetween. The electronic device 100 may match a first straight line passing through a first anterior tooth point and the first center point included in the first plane data with a second straight line passing through a second anterior tooth point and the second center point included in the second plane data. By matching the above-described two straight lines while the center points and normal vectors of the two pieces of plane data match each other, the electronic device 100 may match the directions of vectors having the center points as starting points and the anterior tooth points as ending point in the two planes. Accordingly, the electronic device 100 may match the plane data corresponding to the virtual occlusal plane with the plane data corresponding to the occlusal plane of the subject's oral cavity. Reference numeral 810 in FIG. 8 shows the result of matching the plane data corresponding to the virtual occlusal surface with the plane data corresponding to the occlusal surface of the subject's oral cavity according to the above-described method.

The electronic device 100 according to an embodiment of the present disclosure may align a three-dimensional scan data set on the virtual occlusal plane by matching the plane data corresponding to the virtual occlusal plane with the plane data corresponding to the occlusal plane of the subject's oral cavity. The electronic device 100 of the present disclosure generates the second plane data corresponding to the occlusal surface of the subject's oral cavity from the three-dimensional scan data set, and thus the electronic device 100 may obtain position information relative to the second plane data with respect to multiple coordinate values included in the three-dimensional scan data set. Therefore, when the first plane data corresponding to the virtual occlusal plane matches the second plane data, the electronic device 100 may align the three-dimensional scan data set on the virtual occlusal plane based on the relative position information between the three-dimensional scan data set and the second plane data.

The electronic device 100 according to an embodiment of the present disclosure may align one of a scan data set corresponding to the maxilla (hereinafter, the “maxillary scan data set”) and a scan data set corresponding to the mandible (hereinafter, the “mandibular scan data set”) on a virtual occlusal plane, and then align the other scan data set. The maxillary scan data set and the mandibular scan data set may each be subsets of the three-dimensional scan data set regarding the subject's oral cavity. The electronic device 100 may align either the maxillary scan data set or the mandibular scan data set on the virtual occlusal plane, and then align the other scan data on the virtual occlusal plane based on position information between the maxilla scan data set and the mandible scan data set. The electronic device 100 may obtain the relative position information between the maxillary scan data set and the mandibular scan data set during the process of scanning the subject's oral cavity to acquire the three-dimensional scan data set 700. For example, the electronic device 100 may generate the second plane data based on the scan data set corresponding to the maxilla and match the second scan data set to the first plane data corresponding to the virtual occlusal plane to align the scan data set corresponding to the maxilla on the virtual occlusal plane, and then additionally align the scan data set corresponding to the mandible based on the relative position information between the aligned maxillary scan data set and the mandibular scan data set. Similarly, the electronic device 100 may align the scan data sets corresponding to the mandible and then align the scan data sets corresponding to the maxilla.

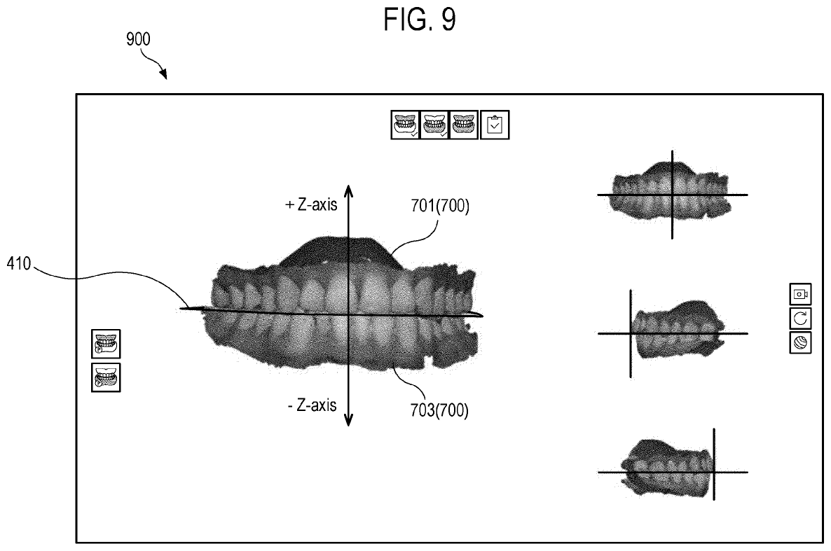

FIG. 9 is an illustrative diagram depicting a result of aligning a three-dimensional scan data set 700 on a virtual occlusal plane 410 by the electronic device 100 according to an embodiment of the present disclosure. Reference numeral 900 in FIG. 9 indicates a display screen for representing alignment results according to an embodiment of the present disclosure. For example, when the electronic device 100 first aligns a maxillary scan data set 701, the maxillary scan data set 701 may be aligned in the (+)z-axis direction of the virtual occlusal plane 410. In this case, a mandibular scan data set 703 may be aligned in the (−)z-axis direction of the virtual occlusal plane 410 based on position information between the mandibular scan data set 703 and the maxillary scan data set 701. In another example, when the electronic device 100 first aligns the mandibular scan data set 703, the mandibular scan data set 703 may be aligned in the (−)z-axis direction of the virtual occlusal plane 410 and the maxillary scan data set 701 may be aligned in the (+)z-axis direction of the virtual occlusal plane 410 based on position information between the maxillary scan data set 701 and the mandibular scan data set 703. In another example, the electronic device 100 may align the maxillary scan data set 701 and the mandibular scan data set 703 on the virtual occlusal plane 410 independently of each other based on the above-described method without using the position information between the maxillary scan data set 701 and the mandibular scan data set 703. In the display screen of FIG. 9, the virtual occlusal plane 410 is represented in the form of a prospective diagram. According to the present disclosure, the three-dimensional scan data set 700 may be aligned on the virtual occlusal plane 410, as shown in FIG. 9.

Hereinafter, another embodiment in which the electronic device 100 of the present disclosure generates plane data from a three-dimensional scan data set and then aligns the plane data on a virtual occlusal plane is described. The electronic device 100 according to an embodiment of the present disclosure may determine whether multiple reference coordinate values include a first coordinate value that is included in a left molar area, a second coordinate value that is included in a right molar area, a third coordinate value that is included in a left area of the oral cavity of the subject 20 and is different from the first coordinate value, and a fourth coordinate value that is included in a right area of the oral cavity of the subject 20 and is different from the second coordinate value. In the present disclosure, the electronic device 100 may determine whether the third coordinate value and the fourth coordinate value are included in the left area and the right area of the oral cavity of the subject 20, respectively, based on a tooth number notation known in the art. For example, the tooth number notation may include FDI notation, Palmer notation, Universal notation, etc. According to a predetermined tooth number notation, when a tooth number corresponding to a specific reference coordinate value is a natural number between 21 and 28 inclusive or between 31 and 38 inclusive, the electronic device 100 may determine that the reference coordinate value is included in the left area of the oral cavity of the subject 20. Further, according to the predetermined tooth number notation when a tooth number corresponding to a specific reference coordinate value is a natural number between 11 and 18 inclusive or between 41 and 48 inclusive, the electronic device 100 may determine that the reference coordinate value is included in the right area of the oral cavity of the subject 20. In the present disclosure, the distinction between left/right, described above, may be reversed depending on the perspective, such as inside or outside of the subject 20. The electronic device 100 may calculate a first midpoint, which is a midpoint of the third coordinate value and the fourth coordinate value. The electronic device 100 may calculate a center point of the second plane data, which is a center point of the first coordinate value, the second coordinate value, and the calculated first midpoint. The electronic device 100 may calculate, as a normal vector of the second plane data, a vector perpendicular to a plane including the first coordinate value, the second coordinate value, and the first midpoint. This will be described in more detail below with reference to FIG. 7B.

FIG. 7B illustrates a method by which the electronic device 100 according to another embodiment of the present disclosure generates plane data from a three-dimensional scan data set. The electronic device 100 may identify a first coordinate value 751 that is included in a left molar area of multiple reference coordinate values determined based on the three-dimensional scan data set 700. The electronic device 100 may identify a second coordinate value 752 that is included in a right molar area of the multiple reference coordinate values determined based on the three-dimensional scan data set 700. The electronic device 100 may identify, among the multiple reference coordinate values determined based on the three-dimensional scan data set 700, a third coordinate value 753 that is included in a left area of a subject's oral cavity and is different from the first coordinate value. The electronic device 100 may identify, among the multiple reference coordinate values determined based on the three-dimensional scan data set 700, a fourth coordinate value 754 that is included in a right area of the subject's oral cavity and is different from the second coordinate value. The electronic device 100 may calculate a first midpoint 755, which is the midpoint of the third coordinate value 753 and the fourth coordinate value 754. The electronic device 100 may calculate, as a center point 771 of the second plane data, a center point of the first coordinate value 751, the second coordinate value 752, and the first midpoint 755. The electronic device 100 may calculate, as a normal vector of the second plane data, a vector perpendicular to a plane including the first coordinate value 751, the second coordinate value 752, and the first midpoint 755.

Subsequently, when the electronic device 100 according to an embodiment of the present disclosure has generated the second plane data based on an example in FIG. 7B as described above, a method for aligning the three-dimensional scan data set on a virtual occlusal plane by using that second plane data will be described.

First, the electronic device 100 may match first plane data corresponding to plane data of the virtual occlusal plane 410 with the second plane data corresponding to the occlusal plane of the subject's oral cavity in the same or similar manner as described above with reference to FIG. 8. That is, the electronic device 100 may match the first center point 431 included in the first plane data with the second center point 771 included in the second plane data. The electronic device 100 may match a first normal vector included in the first plane data with a second normal vector included in the second plane data.

The electronic device 100 according to the present disclosure may match a first straight line passing through the first center point 431 and the first anterior tooth point 433 included in the first plane data with a second straight line passing through the second center point 771 and the first midpoint 755 included in the second plane data. After matching the above-described two straight lines (i.e., the first straight line and the second straight line) with each other, the electronic device 100 may align the three-dimensional scan data set 700 on the second plane data. Specifically, because the second plane data corresponding to the occlusal plane of the subject's oral cavity is generated from multiple coordinate values included in the three-dimensional scan data set, the electronic device 100 may have position information of the multiple coordinate values included in the three-dimensional scan data set relative to the second plane data. Thus, even when at least some of the values included in the second plane data change in the process of matching the first plane data corresponding to the virtual occlusal plane with the second plane data corresponding to the occlusal plane of the subject, the electronic device 100 may align the three-dimensional scan data set on the second plane data based on the position information of the multiple coordinate values included in the three-dimensional scan data set relative to the second plane data.

The electronic device 100 according to the present disclosure may obtain the position information based on the farthest point toward the first midpoint 755 from the second center point 771 of the multiple three-dimensional coordinate values included in the three-dimensional scan data set 700 and the first anterior tooth point 433 included in the first plane data. Specifically, referring back to FIG. 7B to describe the farthest point, the electronic device 100 may determine that a specific three-dimensional coordinate value farthest from the second center point 771 toward the first midpoint 755 among the multiple three-dimensional coordinate values is a farthest point 773. The electronic device 100 may obtain position information based on the farthest point 773 and the first anterior tooth point 433. The obtained position information may include, for example, a coordinate value of the farthest point 773, a coordinate value of the first anterior tooth point 433, a difference between the two coordinate values, a distance between the farthest point 773 and the first anterior tooth point 433, and the like. The electronic device 100 may align the three-dimensional scan data set on the virtual occlusal plane by correcting the three-dimensional scan data set aligned on the second plane data based on the obtained position information. For example, it is assumed that a coordinate value of a farthest point included in the position information is (X1, Y1, Z1) and a coordinate value of the first anterior tooth point is (X2, Y2, Z2). Under the above-described assumption, the electronic device 100 may perform a correction in which a difference value (i.e., (X2−X1, Y2−Y1, Z2−Z1)) between the coordinate value of the first anterior tooth point and the coordinate value of the farthest point is added to each of the multiple three-dimensional coordinate values included in the three-dimensional scan data set. With the correction, the electronic device 100 may match a point corresponding to anterior teeth in the subject's oral cavity with a point corresponding to anterior teeth in the virtual occlusal plane. Thus, the electronic device 100 may align the three-dimensional scan data set on the virtual occlusal plane.

As described above, when the electronic device 100 according to the present disclosure aligns the three-dimensional scan data set on the virtual occlusal plane by using four points including the first coordinate value to the fourth coordinate value, there is an effect that even in case of a three-dimensional scan data set of a subject's oral cavity in which a specific tooth (e.g., incisor, canine, etc.) is missing, the three-dimensional scan data set may be accurately aligned on the virtual occlusal plane. In addition to some embodiments described above with reference to FIGS. 7A and 7B, the present disclosure includes, without limitation, various embodiments of generating second plane data representing the occlusal plane of a subject's oral cavity from multiple reference coordinate values determined from a three-dimensional scan data set regarding the subject.

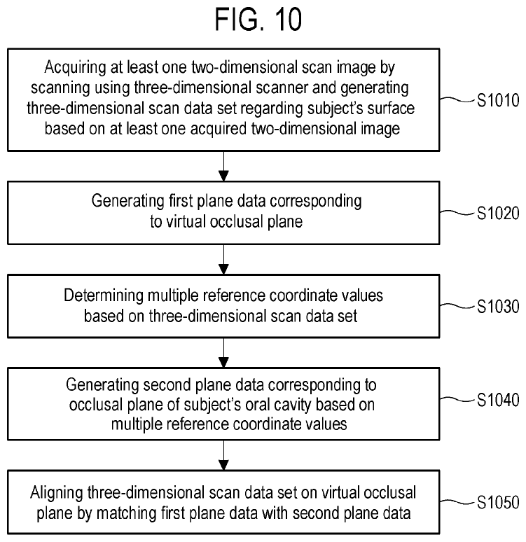

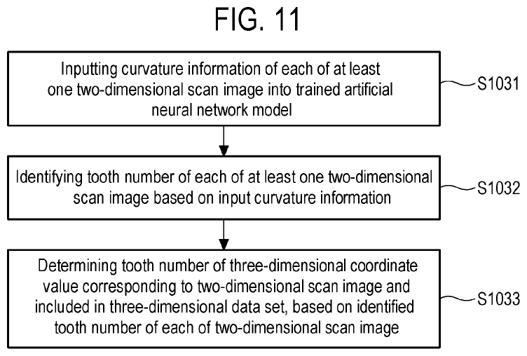

FIG. 10 is a flowchart of operations of an electronic device according to an embodiment of the present disclosure. In operation S1010, the electronic device 100 may acquire at least one two-dimensional scan image by scanning using the three-dimensional scanner 200, and may generate a three-dimensional scan data set regarding a subject's oral cavity based on the at least one acquired two-dimensional scan image. For example, the electronic device 100 may convert the two-dimensional scan image into a point cloud, which is a set of data points having three-dimensional coordinate values, to generate the three-dimensional scan data set regarding the subject's oral cavity. In operation S1020, the electronic device 100 may generate first plane data corresponding to a virtual occlusal plane. The virtual occlusal plane may refer to an imaginary plane for representing the occlusal surface of the subject's teeth as a single plane. The first plane data may include a center point and a normal vector for representing the virtual occlusal plane. The first plane data may further include an anterior tooth point regarding the virtual occlusal plane. The electronic device 100 may receive an input signal from a user via the input device 109 to generate the first plane data corresponding to the virtual occlusal plane. The electronic device 100 may also generate the first plane data corresponding to the virtual occlusal plane, based on a predetermined figure stored in the memory 103. In operation S1030, the electronic device 100 may determine multiple reference coordinate values based on the three-dimensional scan data set. The multiple reference coordinate values may be determined heuristically by the user, or may be determined based on computation of a trained artificial neural network model. An embodiment of determining the multiple reference coordinate values based on the computation of the trained artificial neural network model will be described in more detail below with reference to the flowchart in FIG. 11. In operation S1040, the electronic device 100 may generate second plane data corresponding to the occlusal plane of the subject's oral cavity based on the determined multiple reference coordinate values. To generate the second plane data corresponding to the occlusal plane of the subject's oral cavity, the electronic device 100 may determine whether the multiple reference coordinate values include predetermined types of coordinate values. In an example, the electronic device 100 may determine whether the multiple reference coordinate values include a first coordinate value included in a left molar area, a second coordinate value included in a right molar area, and a third coordinate value included in an anterior tooth area. In another example, the electronic device 100 may determine whether the multiple reference coordinate values include a first coordinate value included in the left molar area, a second coordinate value included in the right molar area, a third coordinate value included in a left canine area, and a fourth coordinate value included in a right canine area. The electronic device 100 may calculate a center point, an anterior tooth point, and a normal vector of the second plane data from the multiple reference coordinate values. In operation S1050, the electronic device 100 may align a three-dimensional scan data set on the virtual occlusal plane by matching the first plane data and the second plane data to each other. After matching the center point and the normal vector of the first plane data with the center point and the normal vector of the second plane data, the electronic device 100 may match a first straight line passing through the center point and anterior tooth point included in the first plane data to a second straight line passing through the center point and anterior tooth point included in the second plane data. Accordingly, the first plane data and the second plane data may be matched to each other, and the three-dimensional scan data set having relative position information with respect to the second plane data may also have relative position information with respect to the first plane data. As a result, the electronic device 100 may align the three-dimensional scan data sets on a virtual occlusal plane.

FIG. 11 is a flowchart of operations of an electronic device according to an embodiment of the present disclosure. The operations illustrated in FIG. 11 may each constitute an embodiment in which, in operation S1030 in FIG. 10, the electronic device 100 determines the multiple reference coordinate values based on the computation of the trained artificial neural network model. In operation S1031, the electronic device 100 may input curvature information of each of the at least one two-dimensional scan image into the trained artificial neural network model. In step S1032, the electronic device 100 may identify a tooth number of each of the at least one two-dimensional scan image based on an output of the artificial neural network model performing a computation based on the input curvature information. In some cases, the electronic device 100 may further input, in step S1031, at least one selected from the group of size information and shape information of each of the at least one two-dimensional scan image into the trained artificial neural network model. In this case, the electronic device 100 may also identify, in operation S1032, a tooth number of each of the at least one two-dimensional scan image based on an output of the artificial neural network model performing a computation based on the input information. In operation S1033, the electronic device 100 may determine, based on the identified tooth number of each two-dimensional scan image, a tooth number of a three-dimensional coordinate value corresponding to the two-dimensional scan image and included in the three-dimensional data set. Since the electronic device 100 generates the three-dimensional scan data set from two-dimensional images, three-dimensional coordinate values included in the three-dimensional scan data set may each be matched to the two-dimensional scan images that served as the basis for the generation thereof. Accordingly, the electronic device 100 may determine that the identified tooth number of each two-dimensional scan image is a tooth number of a three-dimensional coordinate value generated from the two-dimensional scan image. Then, the electronic device 100 may determine multiple reference coordinate values based on the tooth numbers of the multiple three-dimensional coordinate values.

In each of the flowcharts illustrated in the present disclosure, the operations of the method or algorithm according to the present disclosure have been illustrated in a sequential order. However, the operations may be performed not only sequentially but also in parallel or in an order in which the operations may be randomly combined. The description according to this flowchart neither excludes making changes or modifications to the method or algorithm, nor implies that any operation is essential or desirable. In an embodiment, at least some operations may be performed in parallel, iteratively, or heuristically. In an embodiment, at least some operations may be omitted, or other operations may be added.

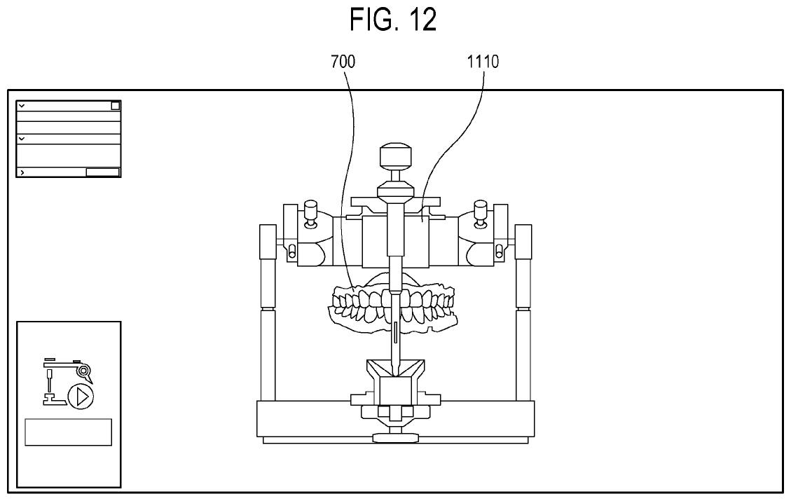

FIG. 12 illustrates an application example of a method for aligning a three-dimensional scan data set on a virtual occlusal plane according to an embodiment of the present disclosure. FIG. 12 illustrates a display screen for performing tasks related to the structure of a subject's oral cavity in a computing environment. The tasks related to the structure of the subject's oral cavity may include, for example, a task of designing a virtual articulator by using CAD or CAM software, a design task for the production of a three-dimensional printed work, and the like. To facilitate the tasks, it is important that a three-dimensional scan data set 700 regarding a subject is correctly placed on a reference plane such as a plane of a virtual articulator 1110. According to the present disclosure, generating plane data about the occlusal plane of the subject's oral cavity and then matching the plane data with plane data about a virtual occlusal plane has the effect of accurately aligning a three-dimensional scan data set representing the subject on the virtual occlusal plane.

Although the method has been described with reference to specific embodiments, the method may also be implemented as computer-readable code on a computer-readable recording medium. The computer-readable recording medium includes all types of recording devices that store data that can be read by a computer system. Examples of computer-readable recording media include ROM, RAM, a CD-ROM, a magnetic tape, a floppy disk, an optical data storage device, and the like. Further, the computer-readable recording medium may be distributed to computer systems connected over a network, so that the computer-readable code may be stored and executed in a distributed manner. Furthermore, functional programs, codes, and code segments for implementing the above embodiments can be readily inferred by programmers skilled in the art to which the present disclosure belongs.

Although the above description provides an example of the technical idea of the present disclosure for illustrative purposes, those skilled in the art to which the present disclosure belongs will appreciate that various modifications and changes are possible without departing from the essential features of the present disclosure. Also, such various modifications and changes should be construed to fall within the scope of the accompanying claims.

1. A method for processing a scan image of a three-dimensional scanner, the method being performed by an electronic device comprising at least one processor and at least one memory which stores instructions to be executed by the at least one processor, the method comprising:
acquiring at least one two-dimensional scan image by scanning of the three-dimensional scanner and generating a three-dimensional scan data set regarding a subject's oral cavity based on the acquired at least one two-dimensional scan image, the three-dimensional scan data set comprising multiple three-dimensional coordinate values;
generating first plane data corresponding to a virtual occlusal plane;
determining multiple reference coordinate values based on the three-dimensional scan data set;
generating second plane data corresponding to an occlusal plane of the subject's oral cavity based on the multiple reference coordinate values; and
aligning the three-dimensional scan data set on the virtual occlusal plane by matching the first plane data with the second plane data.

2. The method of claim 1, wherein the first plane data comprises a first center point, a first anterior tooth point, and a first normal vector, and
wherein the second plane data comprises a second center point, a second anterior tooth point, and a second normal vector.

3. The method of claim 1, wherein determining the multiple reference coordinate values is performed using a trained artificial neural network model, and
wherein the artificial neural network model is trained based on a training data set comprising:
curvature information of each of at least one two-dimensional training image; and
a tooth number corresponding to each of the at least one two-dimensional training image.

4. The method of claim 3, wherein the training data set further comprises at least one selected from the group of:
size information of each of the at least one two-dimensional training image; and
shape information of each of the at least one two-dimensional training image.

5. The method of claim 1, wherein determining the multiple reference coordinate values comprises:
inputting curvature information of each of the at least one two-dimensional scan image into a trained artificial neural network model; and
identifying a tooth number of each of the at least one two-dimensional scan image based on the input curvature information.

6. The method of claim 5, wherein determining the multiple reference coordinate values further comprises inputting at least one selected from the group of size information and shape information of the at least one two-dimensional scan image into the trained artificial neural network model, and
wherein identifying the tooth number is performed additionally based on the input of at least one selected from the group of the size information and the shape information.

7. The method of claim 5, wherein determining the multiple reference coordinate values further comprises determining, based on the identified tooth number of each two-dimensional scan image, a tooth number of a three-dimensional coordinate value corresponding to the each two-dimensional scan image and included in the three-dimensional scan data set.

8. The method of claim 7, wherein determining the multiple reference coordinate values further comprises determining a representative coordinate value of a corresponding tooth based on multiple three-dimensional coordinate values determined to have the same tooth number.

9. The method of claim 1, wherein generating the second plane data comprises:
determining whether the multiple reference coordinate values comprise a first coordinate value included in a left molar area, a second coordinate value included in a right molar area, and a third coordinate value included in an anterior tooth area;
storing the third coordinate value as an anterior tooth point of the second plane data;
calculating, as a center point of the second plane data, a center point of the first coordinate value, the second coordinate value, and the third coordinate value; and
calculating, as a normal vector of the second plane data, a vector perpendicular to a plane comprising the first coordinate value, the second coordinate value, and the third coordinate value.

10. The method of claim 1, wherein aligning the three-dimensional scan data set on the virtual occlusal plane comprises:
matching a first center point included in the first plane data with a second center point included in the second plane data;
matching a first normal vector included in the first plane data with a second normal vector included in the second plane data; and
matching a first straight line passing through the first center point and a first anterior tooth point included in the first plane data with a second straight line passing through the second center point and a second anterior tooth point included in the second plane data.

11. The method of claim 1, wherein generating the second plane data comprises:
determining whether the multiple reference coordinate values comprise a first coordinate value included in a left molar area, a second coordinate value included in a right molar area, a third coordinate value included in a left area of the subject's oral cavity and different from the first coordinate value, and a fourth coordinate value included in a right area of the subject's oral cavity and different from the second coordinate value;
calculating a first midpoint, which is a midpoint of the third coordinate value and the fourth coordinate value;
calculating, as a center point of the second plane data, a center point of the first coordinate value, the second coordinate value, and the first midpoint; and
calculating, as a normal vector of the second plane data, a vector perpendicular to a plane comprising the first coordinate value, the second coordinate value, and the first midpoint.

12. The method of claim 11, wherein aligning the three-dimensional scan data set on the virtual occlusal plane comprises:
matching a first center point included in the first plane data with a second center point included in the second plane data;
matching a first normal vector included in the first plane data with a second normal vector included in the second plane data;
matching a first straight line passing through the first center point and a first anterior tooth point included in the first plane data with a second straight line passing through the second center point and the first midpoint;
aligning the three-dimensional scan data set on the second plane data;
obtaining position information based on the first anterior tooth point and a farthest point from the second center point toward the first midpoint among multiple three-dimensional coordinate values included in the three-dimensional scan data set; and
aligning the three-dimensional scan data set on the virtual occlusal plane by correcting, based on the position information, the three-dimensional scan data set aligned on the second plane data.

13. An electronic device comprising:
a communication circuit communicatively connected to a three-dimensional scanner;
a memory; and
at least one processor,
wherein the at least one processor is configured to:
acquire at least one two-dimensional scan image by scanning of the three-dimensional scanner and generating a three-dimensional scan data set regarding a subject's oral cavity based on the acquired at least one two-dimensional scan image, the three-dimensional scan data set comprising multiple three-dimensional coordinate values;
generate first plane data corresponding to a virtual occlusal plane;
determine multiple reference coordinate values based on the three-dimensional scan data set;
generate second plane data corresponding to an occlusal plane of the subject's oral cavity based on the multiple reference coordinate values; and
align the three-dimensional scan data set on the virtual occlusal plane by matching the first plane data with the second plane data.

14. The electronic device of claim 13, wherein the first plane data comprises a first center point, a first anterior tooth point, and a first normal vector, and
wherein the second plane data comprises a second center point, a second anterior tooth point, and a second normal vector.

15. The electronic device of claim 13, wherein the at least one processor is configured to determine the multiple reference coordinate values by using a trained artificial neural network model, and
wherein the artificial neural network model is trained based on a training data set comprising:
curvature information of each of at least one two-dimensional training image; and
a tooth number corresponding to each of the at least one two-dimensional training image.

16. The electronic device of claim 15, wherein the training data set further comprises at least one selected from the group of:
size information of each of the at least one two-dimensional training image; and
shape information of each of the at least one two-dimensional training image.

17. The electronic device of claim 13, wherein the at least one processor is configured to:
input curvature information of each of the at least one two-dimensional scan image into a trained artificial neural network model; and
identify a tooth number of each of the at least one two-dimensional scan image based on the input curvature information.

18. The electronic device of claim 17, wherein the at least one processor is configured to:
input at least one selected from the group of size information and shape information of the at least one two-dimensional scan image into the trained artificial neural network model, and
identify the tooth number of each of the at least one two-dimensional scan image based on the input curvature information and additionally based on the input of at least one selected from the group of the size information and the shape information.

19. The electronic device of claim 17, wherein the at least one processor is configured to determine, based on the identified tooth number of each two-dimensional scan image, a tooth number of a three-dimensional coordinate value corresponding to the each two-dimensional scan image and included in the three-dimensional scan data set.

20-24. (canceled)

25. A non-transitory computer-readable recording medium that records instructions to be executed on a computer, wherein the instructions are configured to, when executed by at least one processor, cause the at least one processor to:
acquire at least one two-dimensional scan image by scanning of a three-dimensional scanner and generate a three-dimensional scan data set regarding a subject's oral cavity based on the acquired at least one two-dimensional scan image, the three-dimensional scan data set comprising multiple three-dimensional coordinate values;
generate first plane data corresponding to a virtual occlusal plane;
determine multiple reference coordinate values based on the three-dimensional scan data set;
generate second plane data corresponding to an occlusal plane of the subject's oral cavity based on the multiple reference coordinate values; and
align the three-dimensional scan data set on the virtual occlusal plane by matching the first plane data with the second plane data.
